(19) United States
(12) Patent Application Publication
(10) Pub. No.: US 2021/0287764 A1
(43) Pub. Date: Sep. 16, 2021

(54) SYSTEM FOR DETERMINING A SUITABILITY OF AN ACTIVE INGREDIENT TO BE APPLIED TRANSDERMALLY OR TRANSMUCOSALLY AND CORRESPONDING METHOD

(71) Applicant: LTS Lohmann Therapie-Systeme AG, Andernach (DE)

(72) Inventors: Hanshermann Franke, Hamburg (DE); Patrick Mohr, Bad Breisig (DE); René Eifler, Koblenz (DE); Marius Bauer, Andernach (DE)

(73) Assignee: LTS Lohmann Therapie-Systeme AG, Andernach (DE)

(21) Appl. No.: 17/303,519

(22) Filed: Jun. 1, 2021

Related U.S. Application Data
(63) Continuation of application No. 15/118,757, filed on Aug. 12, 2016, now abandoned.

(30) Foreign Application Priority Data
Feb. 25, 2014 (EP) 14156479.9

(51) Int. Cl. G16C 20/50; G16C 20/30; G16C 20/64

(52) U.S. Cl. CPC G16C 20/50; G16B 35/00; G16C 20/64; G16C 20/30

(57) ABSTRACT

A device and a method for determining a suitability of an active ingredient to be applied transdermally or transmucosally. In particular, a system that includes:
at least one input device for inputting at least one designation of an active ingredient;
a database for storing a plurality of data at least relating to the active ingredient;
a computing unit for assessing a suitability of the active ingredient to be applied transdermally and/or transmucosally while taking into consideration data entered via the input device and/or data stored in the database; and
an output device for displaying the assessed suitability.

The invention relates to a system for determining a suitability of an active ingredient for transdermal and/or transmucosal application.

The transdermal application of active ingredients is known. The best known example is the nicotine patch. This is stuck onto the skin where it releases the active ingredient in a controlled manner which is then resorbed via the skin. The active ingredient passes into the blood vessel system without being prematurely broken down in the gastrointestinal tract or in the liver. The concept of the transdermal therapeutic system is also used synonymously

Likewise, the application or administration of active ingredients via the mucous membranes (mucosae) is known. An example of transmucosal application is a lozenge which is taken orally, but is not swallowed but left in the mouth to dissolve. Transmucosal therapeutic systems are preferably used when the area of action of the active ingredient is in the oropharynx (for example local anaesthetics and anti-inflammatory drugs), or when the active ingredient is to pass directly into the bloodstream via the mucous membrane. The latter has two advantages. Firstly, the medication passes very quickly into the bloodstream and secondly, the portal system of the liver can be bypassed in this manner.

In research, a particular problem lies in predicting whether a specific active ingredient can be applied transdermally and/or transmucosally. Expensive clinical studies are often required to establish that a transdermal or a transmucosal application is not indicated for a particular case of use. A further problem is seen in the generation of known models which allow a prediction. The machine processing of corresponding models is computationally intensive and requires a large number of records. The results are inaccurate and difficult to classify.

Based on this prior art, the aim of the present invention is to provide an improved system for determining a suitability of an active ingredient for transdermal or transmucosal application.

This aim is addressed according to the invention by a system according to claim 1 and by a method according to claim 10.

Especially, the aim is addressed by a system for determining a suitability of an active ingredient for transdermal or transmucosal application, the system comprising:
at least one input device for inputting at least a name of an active ingredient;
a database for storing a wide range of data at least relating to the active ingredient;
a computing unit for assessing a suitability of the active ingredient for transdermal or transmucosal application, while considering data input via the input device and/or the data stored in the database;
an output device for displaying the assessed suitability.

An especial advantage of this system is that the suitability can be simply assessed by means of a data processing device, namely by the computing unit. Naturally, a prediction of the suitability of a particular active ingredient for transdermal or transmucosal application cannot replace clinical studies. However, an advantage of the system according to the invention is seen in recognising, at an early stage, candidates who are completely unsuitable for a respective application.

In an embodiment, the computing unit is configured to form a weighted average via a plurality of characteristic numbers in order to assess the suitability. The stated and/or stored data preferably lead to characteristic numbers which indicate the suitability of the active ingredient for transdermal or transmucosal application in individual subject areas. According to the invention, an especially weighted averaging is to be carried out via the plurality of characteristic numbers. For example, it is possible for at least four or six or eight or ten characteristic numbers to be averaged to produce an overall result to indicate the suitability of a particular active ingredient for transdermal or transmucosal application. This plurality of parameters leads to a robust and informative result.

The computing unit can be configured to determine the/a characteristic number, while considering in each case one of the following items of information:
at least one item of information concerning the structural formula of the active ingredient, especially the molecular weight;
at least one item of information concerning the acid strength of the active ingredient, especially the pk_{A} value or pk_{S} value;
at least one item of information concerning the melting point of the active ingredient;
at least one item of information concerning the half-life period;
at least one item of information concerning the water solubility and/or fat solubility of the active ingredient, especially the log P value;
at least one item of information concerning the skin permeability and/or the in vitro permeability (for example permeability rate);
at least one item of information concerning the (oral) bioavailability;
at least one item of information concerning the (oral) dose, especially the (oral) daily dose;
at least one item of information concerning the metabolism;
at least one item of information concerning the skin irritation potential;
at least one item of information concerning the indication; and/or
at least one item of information concerning the dose-response relationship.

These items of information are especially significant for transdermal application, but essentially also for transmucosal application and can be simply and efficiently processed by the computing unit.

At least some of the characteristic numbers can be a selection of numbers from the set of whole numbers. Ultimately, it is possible to map the provided items of information, for example concerning the structural formula, onto an index value or a characteristic number. This characteristic number can preferably be a whole number, thereby producing simple and informative results. In a further preferred embodiment, the characteristic numbers are a selection of integral numbers within a predetermined interval. This set preferably has a relatively low cardinality. For example, this set can contain less than 15 elements or less than 10 elements or less than 7 elements. In one embodiment, the set comprises the following (characteristic) numbers {−3, −2, −1, 0, 1, 2, 3}, in another embodiment it comprises the (characteristic) numbers {−5, −4, −3, −2, −1, 0, 1, 2, 3, 4, 5} or also {0, 1, 2, 3, 4, 5, 6, 7, 8, 9, 10}. Likewise, decimal numbers can be used as characteristic numbers, for example with an accuracy of one decimal place.

The computing unit can be configured, in assessing the suitability of the active ingredient, to consider
the at least one item of information or characteristic number concerning the structural formula of the active ingredient; and/or
the at least one item of information or characteristic number concerning the bioavailability; and/or
the at least one item of information or characteristic number concerning the dose in each case with at least 5% and at most 40%, especially in each case with at least 10% and at most 30%.

As already stated, a weighted averaging is preferably carried out via the characteristic numbers. The structural formula and/or the bioavailability and the dose can be taken into especial consideration in the result concerning the transdermal and/or transmucosal suitability. In one embodiment, the associated characteristic numbers can influence up to a maximum of 30% of the result. The structural formula and the bioavailability and dose preferably find their way into the overall result with at least 5%.

The computing unit can be configured, in assessing the suitability of the active ingredient, to consider
the at least one item of information or characteristic number concerning the acid strength of the active ingredient; and/or
the at least one item of information or characteristic number concerning the melting point of the active ingredient; and/or
the at least one item of information or characteristic number concerning the water solubility and/or fat solubility of the active ingredient; and/or
the at least one item of information or characteristic number concerning the skin permeability; and/or
the at least one item of information concerning the skin irritation potential
   in each case with at least 2% and at most 20%, especially in each case with at least 5% and at most 15%.

The mentioned items of information are important for the overall result. However, it can be advantageous to consider them with a lower weighting. In a preferred embodiment, this weighting is within a range of 3 to 10% per characteristic number. This weighting can result in an especially efficient assessment.

In an embodiment, the database can comprise mapping tables for mapping individual items of information onto characteristic numbers. For example, the mapping tables can state intervals when a particular item of information is assigned to a particular characteristic number. Mapping tables for mapping the
at least one item of information concerning the structural formula of the active ingredient, especially the molecular weight;
at least one item of information concerning the acid strength of the active ingredient, especially the pk_{A} value or pk_{S} value;
at least one item of information concerning the melting point of the active ingredient; and/or
at least one item of information concerning the biological half-life period; and/or
at least one item of information concerning the water solubility and/or fat solubility of the active ingredient, especially the log P value; and/or
at least one item of information concerning the dose
   onto characteristic numbers can be stored in the database.

In a further embodiment, there is a single mapping table which provides mappings onto corresponding characteristic numbers for a plurality of items of information.

The system can comprise at least one measuring device for determining the molecular weight and/or the acid strength and/or the water solubility and/or the melting point. Thus, the system can preferably determine some of the parameters independently and take these into consideration when assessing the suitability of the active ingredient for transdermal or transmucosal application.

Furthermore, the mentioned object is achieved by a method for determining a suitability of an active ingredient according to claim 9.

Especially, the aim is achieved by a method for determining a suitability of an active ingredient for transdermal or transmucosal application, the method comprising the following steps:
acquiring characteristics of the active ingredient;
establishing characteristic numbers in each case for the characteristics of the active ingredient;
calculating a weighted average via the characteristic numbers;
displaying an assessed suitability of the active ingredient using the weighted average.

These steps are preferably carried out by the system which has already been described. Advantages similar to those which have already been described in connection with the device are obtained.

The data which are taken into consideration in the method can comprise:
at least one item of information concerning the structural formula of the active ingredient, especially the molecular weight;
at least one item of information concerning the acid strength of the active ingredient, especially the pk_{A} value or pk_{S} value;
at least one item of information concerning the melting point of the active ingredient;
at least one item of information concerning the half-life period;
at least one item of information concerning the water solubility and/or fat solubility of the active ingredient, especially the log P value;
at least one item of information concerning the skin permeability;
at least one item of information concerning the bioavailability;
at least one item of information concerning the dose, especially the daily dose;
at least one item of information concerning the metabolism;
at least one item of information concerning the skin irritation potential;
at least one item of information concerning the indication; and/or
at least one item of information concerning the dose-response relationship.

The method preferably comprises a step for reading out at least one characteristic number from a/the database. This characteristic number can be determined while considering the mentioned items of information. The database provides intervals in order to map the items of information onto specific characteristic numbers.

The method can comprise a step for comparing the weighted average with a threshold value. This comparison preferably results in a particular active ingredient being considered suitable or unsuitable for transdermal or transmucosal application. In one embodiment, a particular active ingredient is considered as being suitable for transdermal or transmucosal application if the weighted average is greater than a threshold value, especially if it is greater than 0 or 1.

Furthermore, the aim can be addressed by a computer-readable medium with instructions for implementing one of the described methods. The instructions are intended to be carried out on a computer.

Further advantageous embodiments are revealed in the subclaims.

In the following, the invention is described using several embodiments which are described in more detail with reference to drawings.

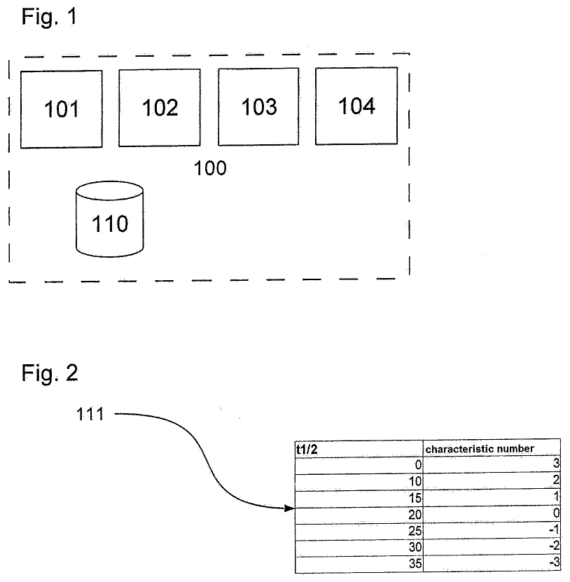

FIG. 1 shows a system for determining a suitability of an active ingredient for transdermal application;

FIG. 2 shows a mapping table for mapping a parameter of the active ingredient onto a respective characteristic number.

FIG. 1 shows a system 100 for determining a suitability of an active ingredient for transdermal application. The system comprises an input device 101 for inputting at least a name of an active ingredient. The input device 101 can also be capable of receiving items of information concerning the structural formula of the active ingredient, the acid strength, the melting point, the half-life period, the water solubility, the fat solubility, the bioavailability, the dose, especially the daily dose, the metabolism, the item of information concerning the skin irritation potential and/or the dose-response relationship. The system 100 also has a computing unit 102 which processes the input data and further data which, for example, are stored in a database 110 to determine the suitability of the active ingredient for transdermal application.

An output device 103 of the system 100 can display the results or can request a user to input data. For example, the user can be requested to provide information concerning the above-mentioned parameters.

In an embodiment, some of these items of information have already been stored in the database 110 and can be retrieved, for example, by entering the name of the active ingredient.

The system 100 optionally has a measuring device 104 which records at least some of the items of information which have already been mentioned and can provide them to be further processed by the computing unit 102. For example, this can be a device for determining the melting point, a spectral analysis device and similar devices.

In an embodiment, after the items of information have been acquired by the computing unit 102, they are mapped onto characteristic numbers. A corresponding mapping can be carried out by a calculation specification.

In an embodiment, the computing unit 102 uses the database 110 to carry out a suitable mapping. For example, an item of information concerning the half-life period can be used in order to establish an associated characteristic number from a half-life period mapping table 111 which is stored in the database 110.

Structurally, the half-life period mapping table 111 is generated so that it has two columns, the first column stating the parameter value of the half-life period “T½” and the second column stating the “characteristic number”. Apart from the header line, the half-life period mapping table 111 comprises seven further lines which are suitable for defining intervals for parameters and for allocating a particular characteristic number thereto. In the illustrated embodiment, the half-life period mapping table 111 is to be interpreted such that characteristic number 3 is assigned to the interval: 0 to less than 10 h(=hours), characteristic number 2 is assigned to the interval: 10 to less than 15 h, characteristic number 1 is assigned to the interval: 15 to less than 20 h, characteristic number 0 is assigned to the interval: 20 to less than 25 h, characteristic number −1 is assigned to the interval: 25 to less than 30 h, characteristic number −2 is assigned to the interval: 30 to less than 35 and characteristic number −3 is assigned to the interval: greater than 35 h.

For example, if an active ingredient has a half-life period of 9 h, the computing unit 102 would assign it characteristic number 3 by means of the half-life period mapping table 111.

The computing unit is configured to establish a characteristic number for each predetermined parameter or for each item of information concerning the active ingredient and to average these characteristic numbers according to a weighting specification. For example, the characteristic number for the structural formula with approximately 20% and the daily dose with approximately 15% can assume a prominent role in assessing the overall result. With a slightly lower weighting (for example approximately 10%), a characteristic number concerning the acid strength of the active ingredient, a melting point, a water solubility and a risk of forming skin irritations (skin irritation potential) can be considered. With an even lower weighting (for example 5%), characteristic numbers which indicate the half-life period, the water solubility and the metabolism can be considered.

For example, if the method according to the invention is applied to the active ingredient nicotine, the following results are obtained. Nicotine has a molecular weight of 62 g/mol, its melting point is −79° C. and its pKA value is 8.9. The half-life period of nicotine is between 1 and 6 h, a log P value of 1.17 being calculated. The required daily dose is usually between 7 and 21 mg and an efficiency of 20-45% is assumed for oral administration.

The following characteristic numbers are obtained for nicotine according to the method of the invention:
structural formula=4;
pKA value=2;
melting point, half-life period, log P value=6;
skin permeability=4;
daily dose, water solubility=−2;
PD data=2;
metabolism and likelihood of skin irritation=0.

Therefore, overall a weighted average of approximately 2.4 is obtained. The weighted average is thus positive, which indicates that a particular active ingredient, in this example nicotine, is suitable for transdermal application. In this embodiment, a weighted average greater than the threshold value of 2 shows that this is a very suitable active ingredient.

In contrast thereto, when the active ingredient Enalapril is assessed, the method according to the invention produces a weighted average of approximately −1. The negative result indicates that this active ingredient is rather unsuitable for transdermal application.

The described method is implemented partly or fully by the computing unit 102.

The described system can likewise be used to establish the suitability of a particular active ingredient for transmucosal application.

### LIST OF REFERENCE NUMERALS

100 system

101 input device

102 computing unit

103 output device

104 measuring device

110 database

111 half-life period mapping table

1.-14. (canceled)

15. A process for ascertaining a suitability of an active ingredient for transdermal or transmucosal application, the process comprising:
receiving an input from a user via a user input device, the input comprising at least a name of the active ingredient;
acquiring a plurality of characteristics of the active ingredient from a database based on the name;
establishing a characteristic number for each individual characteristic of the plurality of characteristics with a computing unit, thereby establishing a plurality of characteristic numbers;
calculating a weighted average of the characteristic numbers with the computing unit; and
displaying an assessed suitability of the active ingredient on an output device based on the weighted average;
wherein the plurality of characteristics comprises:
a first characteristic concerning a structural formula of the active ingredient;
a second characteristic concerning an acid strength of the active ingredient;
a third characteristic concerning a melting point of the active ingredient;
a fourth characteristic concerning a dose of the active ingredient;
a fifth characteristic concerning a skin irritation potential of the active ingredient;
a sixth characteristic concerning at least one of a water solubility and a fat solubility of the active ingredient;
a seventh characteristic concerning a skin permeability of the active ingredient; and
a eighth characteristic concerning a bioavailability of the active ingredient.

16. The process of claim 15, wherein:
the first characteristic is included in the weighted average at a weighting of 5% to 40%;
the second characteristic is included in the weighted average at a weighting of 2% to 20%;
the third characteristic is included in the weighted average at a weighting of 2% to 20%;
the fourth characteristic is included in the weighted average at a weighting of 5% to 40%;
the fifth characteristic is included in the weighted average at a weighting of 2% to 20%;
the sixth characteristic is included in the weighted average at a weighting of 2% to 20%;
the seventh characteristic is included in the weighted average at a weighting of 2% to 20%; and
the eighth characteristic is included in the weighted average at a weighting of 5% to 40%.

17. The process of claim 15, wherein:
the first characteristic is included in the weighted average at a weighting of 10% to 30%;
the second characteristic is included in the weighted average at a weighting of 5% to 15%;
the third characteristic is included in the weighted average at a weighting of 5% to 15%;
the fourth characteristic is included in the weighted average at a weighting of 10% to 30%;
the fifth characteristic is included in the weighted average at a weighting of 5% to 15%;
the sixth characteristic is included in the weighted average at a weighting of 5% to 15%;
the seventh characteristic is included in the weighted average at a weighting of 5% to 15%; and
the eighth characteristic is included in the weighted average at a weighting of 10% to 30%.

18. The process of claim 15, further comprising:
measuring at least one of the characteristics with a measuring device.

19. The process of claim 18, wherein the measured characteristic comprises at least one characteristic selected from the group consisting of a molecular weight of the active ingredient, the acid strength of the active ingredient, the water solubility of the active ingredient, and the melting point of the active ingredient.

20. The process of claim 18, wherein the measuring device comprises a device for measuring the melting point of the active ingredient.

21. The process of claim 18, wherein the measuring device comprises a spectral analysis device.

22. A system for determining a suitability of an active ingredient for transdermal or transmucosal application, comprising:
at least one input device configured to receive an input by a user of at least a name of the active ingredient;
a database comprising data relating to the active ingredient;
a computing unit configured to assess a suitability of the active ingredient for transdermal or transmucosal application, while considering data input via the input device, the data stored in the database, or a combination of the two; and
an output device configured to display the assessed suitability;
wherein the computing unit is configured to form a weighted average using a plurality of characteristic numbers to assess the suitability; and
wherein the computing unit is configured to establish the characteristic numbers while considering in each case at least one item of information selected from the group consisting of:
an item of information concerning a structural formula of the active ingredient;
an item of information concerning a molecular weight of the active ingredient;
an item of information concerning an acid strength of the active ingredient;
an item of information concerning a melting point of the active ingredient;
an item of information concerning a dose of the active ingredient;
an item of information concerning a skin irritation potential of the active ingredient;
at least one item of information concerning an indication of the active ingredient;
an item of information concerning a description of a dose-response relationship of the active ingredient;
an item of information concerning a half-life period of the active ingredient;
an item of information concerning a water solubility, fat solubility, or both, of the active ingredient;
an item of information concerning a skin permeability of the active ingredient;
an item of information concerning a bioavailability of the active ingredient; and
an item of information concerning a metabolism of the active ingredient.

23. The system according to claim 22, wherein the computing unit is configured to establish characteristic numbers while considering in each case at least one item of information selected from the group consisting of:
an item of information concerning a half-life period of the active ingredient;
an item of information concerning a water solubility, fat solubility, or both, of the active ingredient;
an item of information concerning a skin permeability of the active ingredient;
an item of information concerning a bioavailability of the active ingredient; and
an item of information concerning a metabolism of the active ingredient.

24. The system of claim 22, wherein at least some of the characteristic numbers are a selection of whole numbers or of numbers with a decimal place.

25. The system of claim 22, wherein the computing unit is configured, when assessing the suitability of the active ingredient, to take into consideration at least one item of information selected from the group consisting of:
the item of information or characteristic number concerning the structural formula of the active ingredient; and
the item of information or characteristic number concerning the dose, where the dose has at least 5% and at most 40% of the active ingredient.

26. The system of claim 22, wherein the computing unit is configured, when assessing the suitability of the active ingredient, to take into consideration at least one item of information selected from the group consisting of:
the item of information or characteristic number concerning the acid strength of the active ingredient;
the item of information or characteristic number concerning the melting point of the active ingredient; and
the item of information concerning the skin irritation potential of the active ingredient.

27. The system of claim 22, wherein the database stores mapping tables for mapping, onto in each case at least one characteristic number, at least one item of information selected from the group consisting of:
an item of information concerning a structural formula of the active ingredient;
an item of information concerning an acid strength of the active ingredient;
an item of information concerning a melting point of the active ingredient; and
an item of information concerning a dose of the active ingredient.

28. The system of claim 22, further comprising: at least one measuring device configured to determine a molecular weight, an acid strength, a water solubility, a melting point, or a combination thereof, of the active ingredient.

29. A method for determining a suitability of an active ingredient for transdermal or transmucosal application, comprising the steps:
acquiring characteristics of the active ingredient based on an input by a user of at least a name of the active ingredient;
establishing characteristic numbers in each case for the characteristics of the active ingredient;
calculating a weighted average via the characteristic numbers; and
displaying an assessed suitability of the active ingredient using the weighted average;
wherein the characteristics of the active ingredient comprise at least one item of information selected from the group consisting of:
an item of information concerning a structural formula of the active ingredient;
an item of information concerning a molecular weight of the active ingredient:
an item of information concerning an acid strength of the active ingredient;
an item of information concerning a melting point of the active ingredient;
an item of information concerning a dose of the active ingredient;
an item of information concerning a skin irritation potential of the active ingredient;
an item of information concerning an indication of the active ingredient;
an item of information concerning a description of a dose-response relationship of the active ingredient;
an item of information concerning a half-life period of the active ingredient;
an item of information concerning a water solubility, fat solubility, or both, of the active ingredient; and
an item of information concerning a skin permeability of the active ingredient:
an item of information concerning a bioavailability of the active ingredient; and
an item of information concerning a metabolism of the active ingredient.

30. The method according to claim 29, wherein the characteristics of the active ingredient comprise at least one item of information selected from the group consisting of:
an item of information concerning a half-life period of the active ingredient;
an item of information concerning a water solubility, fat solubility, or both, of the active ingredient;
an item of information concerning a skin permeability of the active ingredient;
an item of information concerning a bioavailability of the active ingredient; and
an item of information concerning a metabolism of the active ingredient.

31. The method according to claim 29, wherein the step of acquiring characteristics of the active ingredient comprises:
determining at least one of the characteristics by at least one measuring device.

32. The method according to claim 29, further comprising: reading out, from a database, at least one characteristic number that is assigned to at least one item of information selected from the group consisting of:
an item of information concerning a structural formula of the active ingredient;
an item of information concerning an acid strength of the active ingredient;
an item of information concerning a melting point of the active ingredient; and
an item of information concerning a dose.

33. The method according to claim 29, further comprising:
comparing the weighted average with a threshold value, where the active ingredient is determined as suitable and indicated for transdermal or transmucosal application if the weighted average is greater than the threshold value.

34. A computer-readable medium comprising:
instructions that, when carried out by a computer, implement the method according to claim 29.
